(19) United States
(12) Patent Application Publication
(10) Pub. No.: US 2019/0223844 A1
(43) Pub. Date: Jul. 25, 2019

(54) APPARATUS FOR CAPTURING BODILY WASTE MATTER

(71) Applicant: Baker George Group Limited, London (GB)

(72) Inventors: George James Baker ABOAGYE, London (GB); Neil Bentley CAMMISH, Greater Manchester (GB)

(73) Assignee: Baker George Group Limited, London (GB)

(21) Appl. No.: 16/089,515

(22) PCT Filed: Mar. 31, 2017
(86) PCT No.: PCT/GB2017/050920

§ 371 Date: Sep. 28, 2018

(30) Foreign Application Priority Data
Mar. 31, 2016 (GB) 1605526.1

(51) Int. Cl. A61B 10/00

(52) U.S. Cl. CPC A61B 10/0038; A61B 10/007

(57) ABSTRACT

An apparatus for capturing bodily waste matter within a toilet bowl, the apparatus comprising: a surface adapted to fit within a toilet bowl so as to releasably capture bodily waste matter within the toilet bowl; and a sealable aperture arranged to provide an outlet for release of bodily waste matter captured within the toilet bowl.

The present invention relates to capturing bodily waste matter. More specifically, the invention relates to capturing human bodily waste matter within a toilet bowl. In particular, the invention provides an apparatus and system for capturing bodily waste matter within a toilet bowl so as to be able to measure parameters of and/or record the captured bodily waste matter. A method of recording a measured parameter of bodily waste matter captured within a toilet bowl for a particular user is also provided.

In a healthcare setting it is often necessary to monitor a patient's urine output, and sometimes a patient's stool output, to help understand their ‘fluid-in’ and ‘fluid-out’. For example, when a patient is undergoing chemotherapy it is necessary to monitor the function of the patient's kidneys, as many chemotherapy drugs can cause renal damage. Measuring the urine output of the patient can act as an indicator as to whether or not this has happened. Additionally, the chemical content of the urine or stool can be used to determine whether the patient's kidneys (or other bodily functions) are functioning correctly.

Existing methods of measuring urine volume include using a container held by the patient and a dipstick. Existing methods of measuring stool mass typically require the patient to defecate onto a small tray. The tray is then taken away by the nurse for analysis. Lab tests may then be necessary to determine the chemical composition of the fluid and/or stool. Capturing urine and stool in this way can be awkward, labour-intensive, messy and unhygienic for the patient.

According to one aspect of the invention there is provided an apparatus for capturing bodily waste matter within a toilet bowl, the apparatus comprising: a surface adapted to fit within a toilet bowl so as to releasably capture bodily waste matter within the toilet bowl; and a sealable aperture arranged to provide an outlet for release of bodily waste matter captured within the toilet bowl.

By providing the apparatus with a sealable aperture, the apparatus can be left in situ in a toilet bowl so that the toilet can continue to be used is a conventional sense, by anyone, until such time that it is desired to capture a user's bodily waste matter in the toilet bowl at which time the aperture can be sealed, for example wherein the bodily waste matter is captured after being deposited into the toilet bowl.

The surface may be arranged to extend across the cross-sectional area of a toilet bowl so as to cover (and, optionally, to provide a fluid-tight seal across) said cross-sectional area, such that, when fitted within a toilet bowl, the surface seals the toilet bowl and prevents captured bodily waste from being released from the toilet bowl (for example, into the water trap of the toilet, to be flushed away) when the aperture is sealed. The surface may be arranged to be in contact with the toilet bowl at all points around the entire perimeter of the surface.

Preferably, the apparatus is arranged to sit within (or inside) a toilet bowl rather than to rest on the rim of the toilet bowl, for example. The apparatus may be supported by the toilet seat fixture, or may share such a fixture, which fixture attaches a toilet seat to a toilet, above the toilet bowl.

The surface may be substantially planar, for example wherein the apparatus is a substantially flat plate. The surface may be flat and/or substantially level. Preferably, the entire surface is level. As used herein the term “level” preferably connotes being parallel to the water in a toilet bowl. Alternatively, the surface may be inclined or sloped.

Alternatively, the surface may be arranged to provide a receptacle for capturing bodily waste matter, for example within the receptacle itself. The receptacle may be shaped, at least in part, to conform generally to the internal shape or surface of a toilet bowl.

Optionally, the surface or receptacle may be arranged to extend over at least 20% to at least 60%, preferably at least 70%, and more preferably at least 80% of the area within a toilet bowl bound by a rim or a seat of the toilet.

One or more openings may be provided in the surface, the one or more openings being arranged to allow flush water to enter the receptacle when the apparatus is fitted within a toilet, preferably wherein the one or more openings are arranged to direct (or divert) flush water into the receptacle. Preferably, the surface is configured to be held substantially bodily static relative to the toilet bowl during the release of bodily waste matter through the aperture.

The aperture may be arranged in the surface such that captured bodily waste matter can be released through the aperture by way of gravitational force acting on it when the apparatus is fitted within a toilet bowl. Preferably, the aperture is arranged in the apparatus so as to be in a position directly above the toilet bowl when the apparatus is in situ (for example, when the apparatus is fitted within a toilet bowl), and more preferably directly above the water within the toilet bowl, for example above the neck (or “throat”) of the toilet bowl.

Preferably, the aperture is no larger than the surface area of the water in the toilet bowl. Preferably, the apparatus is arranged to be positioned within the toilet bowl such that, upon flushing the toilet, the flush water can enter the apparatus thereby to help remove any of its contents, for example into the toilet bowl.

The apparatus may further comprise means for sealing the aperture (“sealing means”). The means for sealing the aperture may comprise a member configured to be moveable relative to the aperture such that it can releasably seal the aperture, preferably providing a fluid-tight seal. The member may thereby provide a removable closure across the aperture, to releasable seal the aperture (to the toilet bowl). The means for sealing the aperture may be a rubber or plastic member, for example, and may be arranged to run in a groove, or along a guide rail or track, for example. The means for sealing may be a seal.

The means for sealing the aperture may comprise one of: a gate, preferably a knife gate; a hinged flap or door; a valve; and/or a plug. The means for sealing the aperture may be arranged such that it can be controlled remotely to seal or unseal the aperture. A motor may be arranged to move the means for sealing the aperture, so as to seal or unseal the aperture.

The apparatus may further comprise a leadscrew arrangement connecting the motor to the means for sealing the aperture, wherein the leadscrew arrangement is configured to be actuated by the motor, whereby to move the means for sealing the aperture relative to the aperture, preferably either to seal or unseal the aperture.

The means for sealing the aperture may comprise a hinged flap or door, the apparatus further comprising a pivot arrangement connected to the flap or door and configured to be pivotally actuated by the motor, whereby to move the flap or door relative to the aperture, preferably either to seal or unseal the aperture.

The apparatus may be arranged such that, when fitted within a toilet bowl, the means for sealing the aperture moves to an unsealed position when a flush event of the toilet is detected, preferably wherein said detection and/or movement is performed without user input, for example automatically.

The apparatus may further comprise means for mounting the apparatus to the toilet (“mounting means”), preferably wherein the means for mounting the apparatus is arranged to cooperate and/or couple with the mounting of a seat of the toilet onto a rim of the toilet. Preferably, the means for mounting suspends at least part of the apparatus, preferably the surface, within the toilet bowl, preferably such that the apparatus is not in contact with the toilet bowl (or at least such that the toilet bowl does not support the weight of the apparatus, for example). The means for mounting the apparatus to the toilet may be a mounting or coupling formation.

The means for mounting the apparatus to the toilet may be arranged to rest on the surface of the toilet, and preferably only on the surface of the toilet bowl.

The means for mounting the apparatus is arranged to support the apparatus within the toilet bowl in a spaced arrangement from the walls of the toilet bowl, such that the entire weight of the apparatus is supported by the means for mounting the apparatus.

The surface may be arranged to be detachable from the means for mounting the apparatus such that the surface can be removed from the toilet bowl while containing captured waste matter without having to unmount the apparatus.

The apparatus may further comprise means for measuring a parameter of captured bodily waste matter (“measuring means”). The means for measuring a parameter may be arranged to measure the mass of captured waste matter, and optionally may comprise at least one or more component(s) selected from a group including: a load cell; a strain gauge; a spring scale; a hydraulic scale; and a pneumatic scale. The means for measuring a parameter of captured bodily waste matter may be a sensor (e.g. a mass/weight sensor), gauge, monitor and/or detector.

The means for measuring the mass of captured bodily waste material (which, preferably, is or is comprised of the means for measuring a parameter of captured bodily waste matter) may be arranged to support the surface within the toilet bowl in a spaced arrangement from the walls of the toilet bowl so as to measure the mass of captured bodily waste matter, preferably wherein the means for measuring the mass is integral with the means for mounting the apparatus as described above. The means for measuring the mass of captured bodily waste material may be a sensor (e.g. a mass/weight sensor) and/or weight/mass gauge,

The means for measuring may be (further) arranged to analyse a biological, physical and/or chemical property of captured bodily waste matter. The (further) means for measuring may be at least one of: means for analysing proteins; means for analysing Haemoglobin; means for analysing myoglobin; means for analysing Carbohydrate metabolism disorder; means for analysing Glucose; means for analysing Ketone; means for analysing liver disease; means for analysing haemolytic disorders; means for analysing Bilirubin; means for analysing infections; means for analysing Nitrites; means for analysing Leukocytes; means for analysing kidney disease; means for measuring density or solute concentration; and means for analysing urinary tract disease. The further means for measuring may be a sensor (e.g. a biosensor), detector, gauge, and/or a monitor.

The apparatus may be arranged to measure a parameter of bodily waste matter captured within the toilet bowl, preferably prior to the captured bodily waste matter being released from the apparatus, for example via the aperture.

The apparatus may further comprise means for identifying a type of bodily waste matter deposited into the toilet bowl, for example by means of chemical and/or physical analysis. The means for identifying a type of bodily waste matter deposited into the toilet bowl may be a sensor, detector, gauge, and/or a monitor.

According to another aspect of the invention there is provided a toilet comprising an apparatus as described above, preferably wherein the apparatus is fixedly fitted within the toilet bowl.

According to another aspect of the invention there is provided a system for capturing bodily waste matter in a toilet bowl of a toilet, comprising: means for receiving an input for identifying a user of the toilet; and means for capturing bodily waste matter deposited into a toilet bowl.

The means for receiving an input for identifying a user of the toilet may be a user identification device, for example comprising an input for receiving a user identification, memory for storing the user identification, and/or a processor for processing the user identification. The means for capturing bodily waste matter deposited into a toilet bowl may be a surface for capturing bodily waste, for example any surface, mesh, vessel and/or container that is capable of intercepting, storing and/or containing bodily waste.

The means for receiving an input may be arranged to control an outlet of the means for capturing bodily waste matter, preferably between an open and closed position. The means for receiving an input may comprise a device for receiving an input from a user, preferably wherein the input is provided via at least one of: a touchscreen; a key pad; an electronic fob; a magnetic strip; an RFID reader; barcode or matrix codes; and user-selection from a pre-defined list of users presented. The means for receiving an input may be an input, whether mechanical or electrical.

The means for capturing bodily waste matter preferably comprises an apparatus as described above. The system may further comprise means for measuring a parameter of captured bodily waste matter, preferably as described above. The system may further comprise means for storing the measured parameter in a database on a record associated with the identified user. The means for storing the measured parameter may be a data storage device, for example memory.

According to another aspect of the present invention there is provided a kit of parts, comprising an apparatus as described above and one or more adaptors configured to enable the apparatus to be fitted within one or more different toilet bowls having different dimensions and/or shapes. The kit of parts may also comprise a means for receiving an input for identifying a user of the apparatus.

According to another aspect of the present invention there is provided a method of recording a measured parameter of bodily waste matter captured within a toilet bowl for a particular user, comprising: identifying a user of an apparatus for capturing bodily waste matter in a toilet bowl; receiving a measured parameter associated with bodily waste matter captured in the apparatus; and storing said measured parameter in a memory, preferably in a database on a record associated with the identified user.

The method may further comprise processing the received measured parameter to determine a further parameter. The method may further comprise sealing an aperture of the apparatus upon identification of a user prior to the user depositing bodily waste matter into the toilet bowl, preferably wherein said user is an authorised user. The apparatus may be an apparatus as described above.

There is also provided an apparatus, system and toilet substantially as herein described with reference to the accompanying figures.

As used herein, the term “toilet” preferably connotes a fixed receptacle into which a person may urinate or defecate, typically consisting of a large bowl (“toilet bowl”) fluidly connected to a system for flushing away the deposited bodily waste matter out of the toilet bowl and into a sewer, or more generally to any system of disposing of the waste. Furthermore, the term “toilet” preferably connotes a lavatory, water closet (WC), privy, latrine, outhouse, or urinal, for example. As used herein, the term “toilet bowl” preferably connotes the receptacle of the toilet that receives waste bodily matter, for example below the rim of the toilet. As used herein, the term “bodily waste” preferably connotes urine, stool/faeces, and/or vomit, which are preferably deposited into a toilet for disposal.

The invention extends to methods and/or apparatus substantially as herein described with reference to the accompanying drawings.

As used herein, means plus function features may be expressed alternatively in terms of their corresponding structure. Any feature in one aspect of the invention may be applied to other aspects of the invention, in any appropriate combination. In particular, apparatus aspects may be applied as method aspects, and vice versa. The invention may comprise any feature as described, whether singly or in any appropriate combination. Furthermore, features implemented in hardware may generally be implemented in software, and vice versa. Any reference to software and hardware features herein should be construed accordingly.

One or more exemplary embodiments of the invention will now be described with reference to the accompanying drawings, in which:

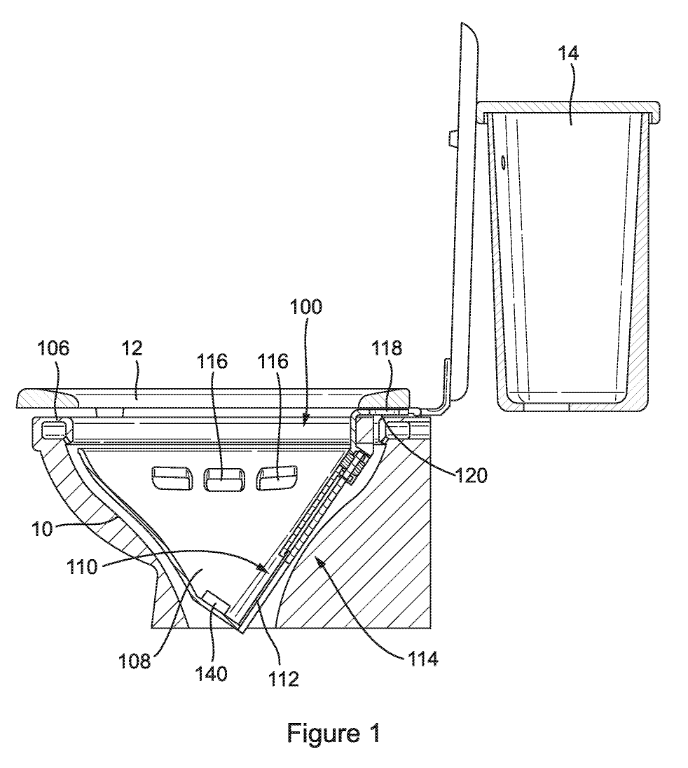

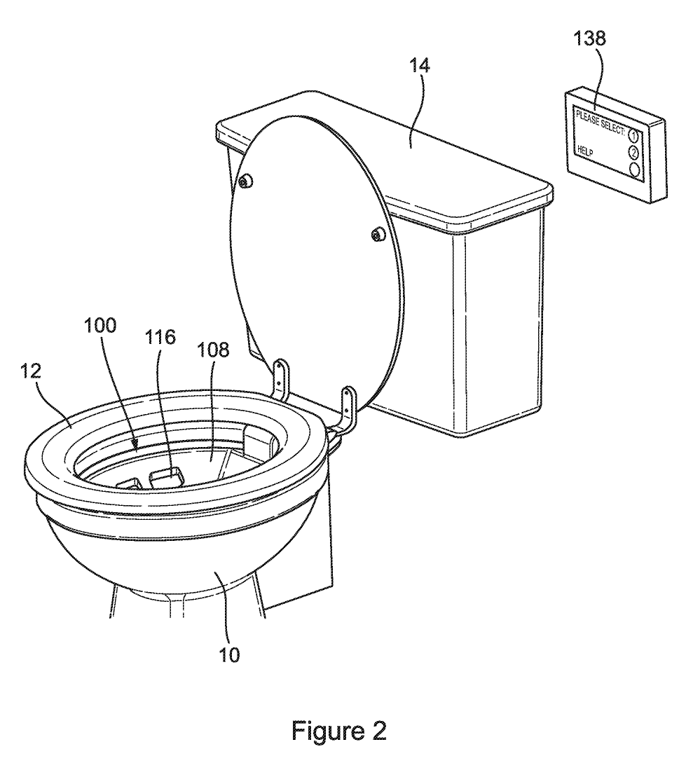

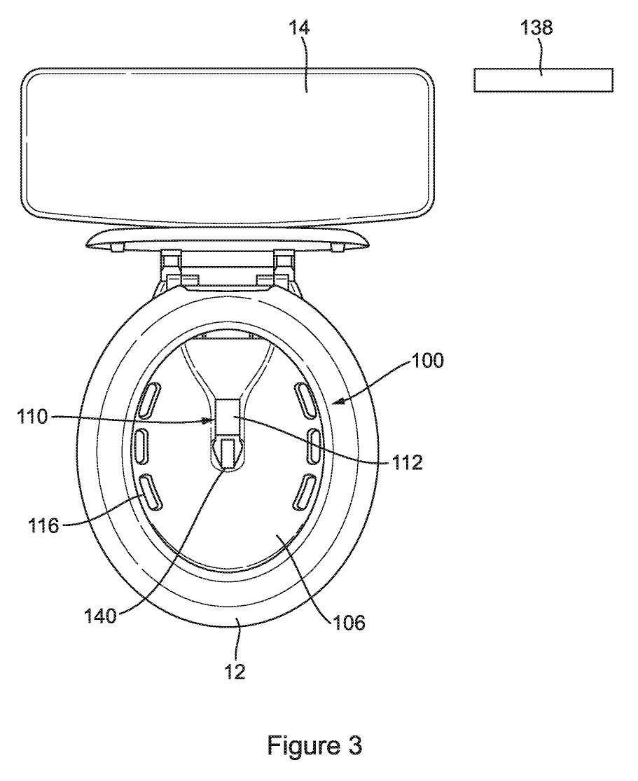

FIGS. 1 to 3 show different views of an apparatus for capturing bodily waste matter in a toilet bowl;

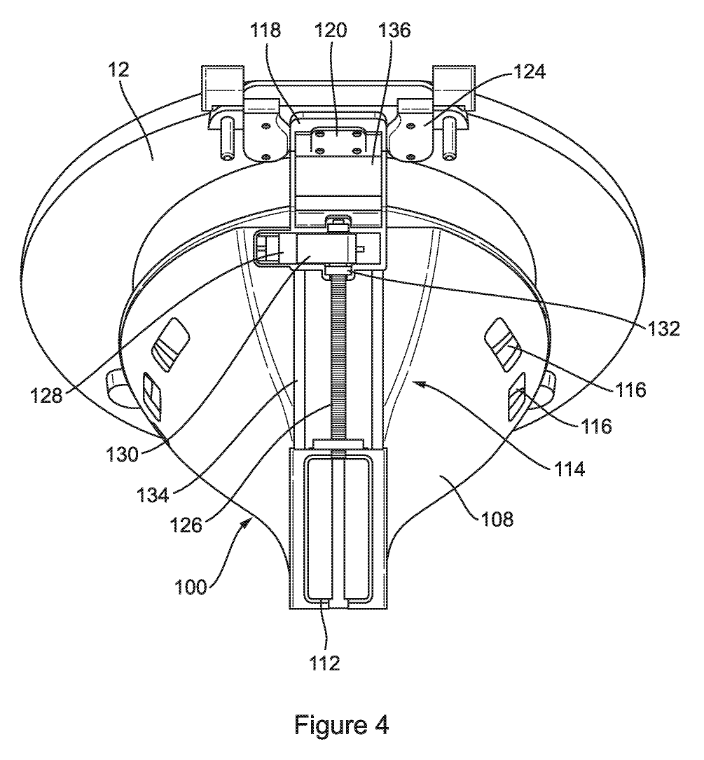

FIG. 4 shows the apparatus of FIGS. 1-3 having a knife gate mechanism arranged to releasably seal an aperture in the apparatus;

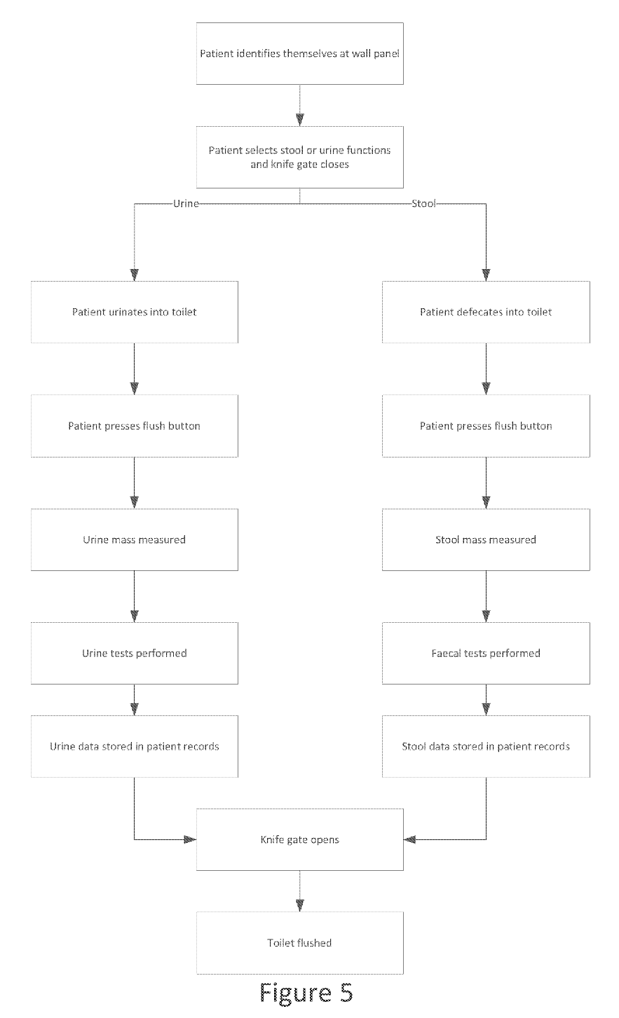

FIG. 5 provides a method of capturing bodily waste matter and determining one or more parameters thereof; and

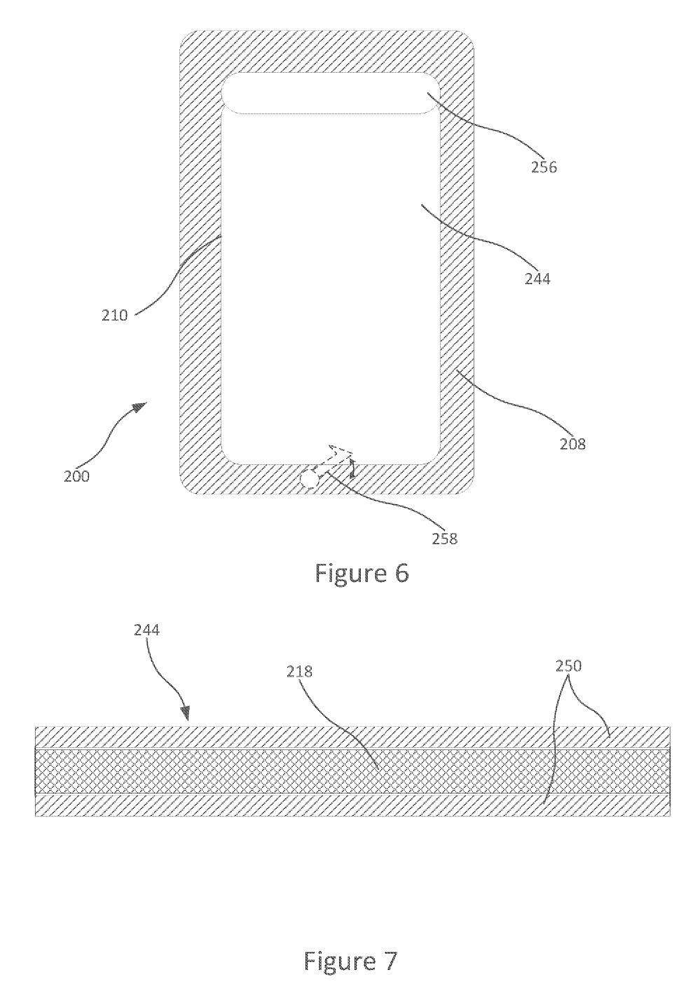

FIGS. 6 to 8 show different views of another apparatus for capturing bodily waste matter in a toilet bowl.

As shown in FIG. 1, a western-style toilet typically comprises a toilet bowl (or “toilet pan”) 10, a seat 12 and a cistern 14. A water trap (not shown) is typically provided at the bottom of the toilet bowl, into which bodily waste matter deposited into the toilet, either from a sitting or standing position, generally ends up, prior to being flushed away. The toilet bowl 10 is provided with a rim 106 that supports the toilet seat 12.

FIG. 1 shows an example of an apparatus 100 having a surface 108 adapted to fit within the toilet bowl 10 so as to capture waste matter deposited into the toilet bowl 10. In this example, the surface 108 is arranged as a receptacle 108. The receptacle 108 depends into the bowl 10 from the bowl rim 106, forming an internal enclosure in the toilet bowl 10. The receptacle 108 is suspended within the bowl 10 to a depth that is above the water trap (i.e. the level to which the toilet bowl 10 is filled with water), thereby preventing buoyant support of the receptacle 108.

An aperture 110 is provided at the base of the receptacle 108. The aperture 110 is arranged to be sealed by movable barrier 112 in the form of a gate. The gate 112 is arranged to be actuated between an open position (in which the aperture 110 is unsealed/not sealed) and closed position (in which the aperture 110 is sealed). The aperture 110 is arranged to provide an outlet for release of bodily waste matter captured within the toilet bowl 10. It should be noted that the terms aperture and outlet may be used interchangeably herein.

The aperture 110 is opened and closed by means of the gate 112 and the gate actuating mechanism 114 to allow human waste to be collected for measurement and then released, by means of gravity and/or flushing, once measured.

The receptacle 108 is shaped generally to conform to the shape of the toilet bowl 10 so that human bodily waste deposited into the toilet bowl 10 is captured by the receptacle 108. In the embodiment shown in FIG. 1, the front and sides of the receptacle 108 are curved to conform approximately with the shape of the inside of the toilet bowl 10, while the rear of the receptacle 108 is straight in order to accommodate a gate actuating mechanism 114. The receptacle 108 is shaped to direct liquids (whether from a user or from the toilet itself) entering the receptacle 108 towards the gate 112, and hence the aperture 110.

Several openings 116 are provided on the sides of the receptacle 108. These are positioned towards the top of the receptacle 108, and below the height of the rim 106. These openings 116 allow water required for flushing the toilet to enter the receptacle 108, and to flush away human waste matter, optionally after having taken a measurement associated with the human waste within the receptacle 108. The openings 116 also allow liquid and/or solids to escape from the receptacle 108 in the event of overflow.

In this apparatus 100, a weighing arrangement 118, in the form of a strain gauge, is used to determine the weight of the receptacle 108 and its contents. The receptacle 108 is therefore suspended in the toilet bowl 10 via the strain gauge 118.

The receptacle 108 is coupled to, and preferably supported by, the strain gauge 118 that is in turn mounted to the toilet. The strain gauge 118 is mounted on the receptacle 108, using a fitting 120. In the embodiment shown in FIG. 1, the fitting 120 is attached to the rim 106 at the rear of the bowl 10, in between the mounting hinges 124 of the toilet seat 12. However, the strain gauge 118 may alternatively be arranged to be mounted from any position(s) around the rim 106 or toilet, or beyond the toilet.

As mentioned above, the gate 112 is arranged to be actuated between a sealed “closed” position (when a user is depositing waste) and an unsealed “open” position (when the waste is to be evacuated, or released, from the receptacle 108), for example when the toilet is flushed (or simply by the force of gravity). In this way, the receptacle 108 remains sealed when the gate 112 is closed, and allows for effective collection and measurement of the user's waste matter. In turn, the waste matter is allowed electively to be flushed away, thereby cleaning the receptacle 108, once the user has finished depositing their waste and desired parameters of the waste matter have been measured.

A watertight seal is formed between the gate 112 and the aperture 110 when the gate 112 is sealed “closed”. This prevents liquid waste from draining from the receptacle 108 until the gate 112 is unsealed “opened”. The watertight seal is achieved by providing a face seal (not shown). The seal is only contacted during the final part of the gate 112 closing.

FIG. 4 illustrates in more detail the mechanism of the gate 112, which in this example is a knife gate. The gate 112 is provided as a movable gate operated by means of the gate actuating mechanism 114, which comprises a leadscrew 126 attached to the gate 112. The gate actuating mechanism 114 further comprises a transverse mounted DC motor 128 and gearbox 130 for actuating the leadscrew 126. The leadscrew 126 is constrained by thrust bearings 132 to prevent premature failure of the gearbox 130. A series of rollers are attached to the gate 112, which allow it to slide up and down along a pair of substantially parallel guide rails 134.

The DC motor 128 and gearbox 130 are controlled by a PCB array 136, which is in communication with the motor. The DC motor 128, gearbox 130 and PCB 136 array are sealed within a watertight enclosure mounted on the outside rear of the receptacle 108. The motor 128 and gearbox 130 are made watertight by applying a resin or polyurethane coating following assembly, or by means of a pre-fitted sealed container. The skilled person will recognise that many other methods are possible.

The PCB 136 monitors the electronic current usage of the DC motor 128. Above a predefined current threshold, the PCB 136 is arranged to cut-off or reverse the DC motor 128. For example, in the event of a blockage of the gate travel, the DC motor 128 would demand current exceeding the predefined threshold. The PCB 136 responds by flagging a fault and stopping or reversing the gate 112 and trying again before flagging the fault to the user.

The PCB 136 is linked to a control panel, shown as a wall panel 138, either wirelessly or by a wired connection. The wall panel 138 is mounted on a surface near the toilet, such as the wall or the cistern 14 of the toilet, such that it is accessible by a user. The wall panel 138 is adapted to be connected to a network, either wirelessly via Bluetooth, Wi-Fi or similar, or via a wired connection. The wall panel 138 is, for example, a touchscreen control panel.

The apparatus 100 further comprises test apparatus 140 for analysing the waste matter deposited in the receptacle 108. The test apparatus 140 is arranged on the internal face of the receptacle 108 and towards its bottom, near to the gate 112, and the test apparatus 140 is exposed in order to contact the contents of the receptacle 108. The test apparatus 140 comprises devices for testing at least one of: density, electrolyte level, disease of the kidneys or urinary tract; protein; haemoglobin and myoglobin; carbohydrate metabolism disorders; glucose; ketone; liver diseases and haemolytic disorder; bilirubin; urobilinogen; nitrites; and/or leukocytes. The test apparatus is in communication with the PCB 136.

The test apparatus 140 preferably comprises a test strip, such as a multi-test strip (for example, the Multistix® urine test strip) that is manually retrievable. In addition, or alternatively, the test apparatus comprises electronic probes (not shown) from which data is manually or remotely retrievable.

The receptacle 108 is arranged so as not to impede conventional operation of the toilet; the toilet is still capable of disposing waste matter in the conventional fashion thanks to the sealable aperture 110 that is arranged to provide an outlet for waste matter.

The receptacle 108 is preferably manufactured from plastic and provided with a smooth and low friction finish for ease of cleaning in order to improve hygiene and usability. The receptacle 108, and all moving parts, are arranged to avoid traps where waste can accumulate or difficult to reach areas during cleaning.

The receptacle 108 is preferably dimensioned to extend over an area of approximately 60% to 10%, and preferably 80%-95%, of the area bound by the rim 106 or seat 12. In this way, the receptacle 108 is arranged to collect waste effectively and ergonomically.

The receptacle 108 may be generally funnel-shaped in order to direct waste matter deposited into the receptacle 108 towards the aperture 110.

The receptacle 108 is arranged in the toilet bowl 10 so that it is only supported by the strain gauge 118, it therefore remains—suspended—clear of the toilet bowl 10. The receptacle 108 is also arranged to remain substantially statically fixed within the toilet bowl 10 (other than to permit an accurate weight reading to be made by the strain gauge).

FIG. 5 illustrates a method of using the weighing and analysis apparatus, which is as follows. When not in use, the gate 112 in the receptacle 108 is available to be either in an open or closed position. Before a user deposits their waste into the receptacle 108, a user identifies themselves by means of the wall panel 138. This is done, for example, with the use of an RFID card, by pre-definition, by inputting an identity number, such as, if in a hospital environment, the user's bed number or patient number. This indicates to a system for operating the weighing and analysis apparatus to whom to attribute data obtained by apparatus.

Following a user identifying themselves to the system, the user may select which bodily function they believe they are about to perform, or have already performed, e.g. urination and/or defaecation. This selection will determine what tests are performed by the system and what data will be recorded.

If the gate 112 in the receptacle 108 was in the open position, it is now closed, sealing the aperture 110 in the receptacle 108. If the gate 112 was already in the closed position, this step is unnecessary.

The user then deposits their bodily waste into the receptacle 108. The user's waste is collected by the receptacle 108. The weight of the deposited waste is measured using the strain gauge 118 supporting the receptacle 108, which feeds the information to the PCB 136, which is available to output this information. In one example, the weight measurement that is recorded is the maximum weight value reached prior to release of the waste from the receptacle 108, prior to when the user flushes the toilet or after a predefined period of time without any change in the weight reading.

A measurement of weight is preferably recorded prior to any toilet paper—or more generally, any foreign, non-body waste matter—being deposited into the receptacle 108 by the user; this is achieved, for example, by taking a measurement as the user rises, or immediately after the user has risen, from the toilet seat 12; to detect this the toilet seat 12 may be provided with a weight or IR sensor which is in communication with the PCB 136.

At the same time, the apparatus 100 initiates test analysis of the waste by means of the test apparatus 140. The results of this analysis, as well as the weight of the deposited waste, are recorded and attributed to the identified user.

If the results from the test apparatus 140 are unusual, the apparatus 100 may be configured to retain—rather than to release—the captured bodily waste, or at least to capture a sample of the waste matter, in order to permit further testing and analysis of the waste matter.

Once the user has finished depositing their waste matter, they may activate a flush, e.g. using a button on the toilet or on the wall panel 138. Alternatively, the mechanism of the toilet is triggered automatically, by means of, for example, a ‘touchfree’ IR sensor.

The gate 112 in the base of the receptacle 108 then opens. The toilet may then be flushed in the normal way, with the flush water entering the receptacle 108 via the openings (e.g. “cut-outs”) 116 provided in the sides of the receptacle 108 and/or via the upper opening of the receptacle 108. The flush water typically cleans the inside surface of the receptacle 108, removing the deposited waste as it is directed out of the outlet 110 by gravitational force and/or by water pressure.

The apparatus 100 may be arranged to trigger any number of flushes at any stage during the use of the apparatus/toilet, for example: once before capturing bodily waste; once after capturing bodily waste in the apparatus 100, but before the waste has been weighed and measured; and/or once more after toilet paper has been deposited into the apparatus/toilet.

If the default position of the gate 112 when not in use is the closed position, the gate 112 will then close. Alternatively, the gate 112 may close once the weight in the receptacle 108 is determined by the strain gauge 118 is approximately zeroed—indicating that no waste matter remains in the receptacle 108.

The default position of the gate 112 when not in use is preferably the open position, and ideally it will remain open until the next instance of a user identifying themselves at the wall panel 138, or similar ID means.

The data recorded by the system, including weight of bodily waste, e.g. fluid output from a user, and the results of any analysis conducted on any bodily waste matter, can be stored by the system. For example, the wall panel 138 may have a storage facility (e.g. memory) for later access and retrieval by, for example, a medical professional. The medical professional may enter a PIN into the wall panel 138 to identify themselves as being authorised to access the data, and thereby be permitted to retrieve individual and/or cumulative urine and stool data for a particular user.

Alternatively, if the wall panel 138 is connected to a data network, the data recorded by the system is sent to a database on the network. This is, for example, a hospital database. The data is then associated with a particular patient, in a user profile, and included on that patient's electronic chart, or similar record.

As human waste is deposited into the toilet bowl 10, whether in liquid or solid form, is collected and measured by the receptacle 108. This is preferable from having to capture such waste matter in a hand-held container, for example. Additionally, the present system does not require the user to aim to deposit waste onto a suspended platform in a particular position, thereby improving usability and ergonomics.

Another example of an apparatus 200 is shown in FIG. 6. In this embodiment, the apparatus 200 has a substantially planar surface 208, for example arranged in the form of a plate arranged to cover the cross-sectional area of the toilet bowl 10 thereby sealing it. The surface 208 is arranged to seal a water trap 248 at the base of a toilet bowl 10 so as to prevent any captured waste matter from being released from the toilet bowl 10 (until such release is required).

An aperture 210 is arranged to provide an outlet for release of captured waste matter in the toilet bowl 10. The aperture 210 provided in the surface 208 of the apparatus 200. A moveable barrier 244 in the form of a hinged flap (e.g. a “trapdoor”) 244 is arranged to seal the aperture 210.

The apparatus 200 is arranged either to be fixed to, or merely rest upon, the inside wall of the toilet bowl 10. The apparatus 200 is suitable for being fixed to the inside of the toilet bowl above the level of the water trap 248. Human waste deposited into the bowl 10 will therefore be captured within the toilet bowl 10 and prevented from entering the water trap 248.

A plunger-like seal (not shown) may be provided to attach the apparatus 200 to the toilet bowl 10. The plunger-like seal is preferably watertight to prevent liquid waste from leaking past the apparatus 200. The trapdoor 244 is lockable in a sealed “closed” position by means of a latch 256 provided on the surface 208.

As shown in FIG. 7, the trapdoor 244 may incorporate a weighing arrangement 218, such as a load cell, strain gauge or a pressure plate, for example, preferably enclosed within a waterproof layer 250, such as plastic. Via this layer 250, the weight of deposited waste matter may be measured by the weighing arrangement 218, which is preferably connected to a control and measuring circuit (not shown) that records measurements made by the material. This control circuit is preferably connected wirelessly or by wires to a wall panel (also not shown) similar to the wall panel 138 in the previously described apparatus 100. The control circuit is preferably arranged to transmit measurement data to the wall panel for storage.

The method of weighing bodily waste in this example is substantially identical to the method described with reference to FIG. 5. An intended user inputs their identification details into the wall panel. Upon identification, the system is arranged to control the operation of the apparatus 200, such as whether to: activate, seal and/or unseal the aperture 210. Furthermore, identification indicates on/under which records (e.g. user profile) the measurements (e.g. measured parameter(s)) obtained by the system are to be stored. The user then deposits their waste matter into the toilet bowl 10. The waste matter is captured within the toilet bowl 10 due to the apparatus 200 preventing it from reaching the water trap 248, and its weight may thus be measured by the weighing arrangement 248 provided within the trapdoor 244.

When the user has finished depositing their waste, they may press a ‘flush’ button on the wall panel, or similar. The measured parameter(s) data is then transmitted by the control circuit to the wall panel.

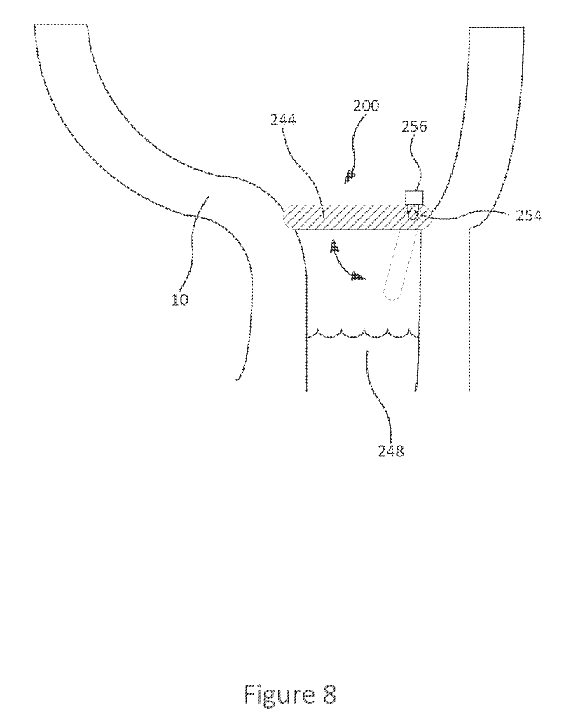

As shown in FIG. 8, the latch 258 may then be released so that the trapdoor 244 is also released, moving to its open position about a pivot 254 (such as a pin or rod, for example) coupled to the surface 208 to unseal the aperture 210. Flushing can then commence, as normal. The data transmitted to the wall panel is stored under the user records associated with the identified user.

Once flushing is complete and/or when a new user requires use of the apparatus 200, the trapdoor 244 may be rotated about the pivot 254 back to its sealed “closed” position by means of a motor (not shown) that raises the trapdoor 244.

In moving to an unsealed “open” position, the trapdoor 244 is, preferably, arranged to hinge downwards, rather than upwards, in order not to trap any bodily waste matter and so as more effectively to release the waste matter.

In another example (not shown), the apparatus 200 may be supported from underneath, for example, by a weighing apparatus.

The apparatus 100, 200 may be arranged to be retrofitted to any toilet bowl 10 by, for example, coupling the apparatus 100, 200 to a toilet bowl 10 by means of the mounting/fitting 120 or sealing the apparatus 100, 200 to, or in, the toilet bowl 10. In order to allow the apparatus 100, 200 to be fitted various shapes and/or sizes of toilet bowl 10, the apparatus 100, 200 may be provided in kit form, including a variety of different shaped adaptors that allow the surface 108, 208 to conform to different shapes of toilet bowl 10 that are available. The surface 108, 208 is, preferably, arranged to be detached from the weighing arrangement 118, 218 (e.g. a strain gauge) and interchanged with another similar surface.

The strain gauge may be replaced with any of: a load cell, a spring scale; and a hydraulic and/or a pneumatic scale, for example.

In another alternative, the aperture/outlet 110, 210 may be sealed by means of: a shutter; a valve; and/or a plug.

It will be understood that the invention has been described above purely by way of example, and modifications of detail can be made within the scope of the invention.

Each feature disclosed in the description, and (where appropriate) the claims and drawings may be provided independently or in any appropriate combination.

Reference numerals appearing in any claims are by way of illustration only and shall have no limiting effect on the scope of the claims.

1. An apparatus for capturing solid and liquid bodily waste matter within a toilet bowl, the apparatus comprising:
a receptacle adapted to fit within a toilet bowl so as to releasably capture solid and liquid bodily waste matter within the toilet bowl,
the receptacle including a sealable aperture arranged to provide an outlet for release of the solid and liquid bodily waste matter captured within the receptacle; and
a weighing arrangement for measuring the mass of the captured solid and liquid bodily waste matter.

2. An apparatus according to claim 1, wherein the receptacle is arranged to extend across the cross-sectional area of a toilet bowl so as to cover said cross-sectional area, such that, when fitted within a toilet bowl, it seals the toilet bowl and prevents the captured solid and liquid bodily waste matter from being released from the toilet bowl when the aperture is sealed.

3. An apparatus according to claim 1, wherein the receptacle is arranged to be in contact with the toilet bowl at all points around the entire perimeter of the receptacle.

4-5. (canceled)

6. An apparatus according to claim 1, wherein the aperture is arranged within the surface such that it is positioned directly above the water trap when the apparatus is fitted within the toilet bowl, whereby to release captured matter directly into the water.

7. (canceled)

8. An apparatus according to claim 1, wherein one or more openings are provided in the receptacle, the one or more openings being arranged to allow flush water to enter the receptacle when the apparatus is fitted within a toilet, preferably wherein the one or more openings are arranged to divert flush water into the receptacle.

9. (canceled)

10. An apparatus according to claim 1, wherein the aperture is arranged in the receptacle such that the captured solid and liquid bodily waste matter can be released through the aperture by way of gravitational force acting on it when the apparatus is fitted within a toilet bowl.

11. An apparatus according to claim 1, further comprising means for sealing the aperture preferably wherein the means for sealing the aperture is arranged to release the solid and liquid bodily waste matter without obstructing the path of the solid bodily waste matter to the aperture.

12. (canceled)

13. An apparatus according to claim 11, wherein the means for sealing the aperture comprises one of: a gate, preferably a knife gate;
a hinged flap or door; a valve; and/or a plug.

14-17. (canceled)

18. An apparatus according to claim 11, wherein the apparatus is arranged such that, when fitted within a toilet bowl, the means for sealing the aperture moves to an unsealed position when a flush event of the toilet is detected, preferably wherein said detection and/or movement is performed without user input, for example automatically.

19-21. (canceled)

22. An apparatus according to claim 1, further comprising means for measuring a parameter of the captured solid and liquid bodily waste matter, preferably wherein the means for measuring a parameter comprises a test apparatus.

23. An apparatus according to claim 22, wherein the means for measuring parameter comprises the weighing arrangement, and the weighing arrangement is at least one of: a load cell; a strain gauge; a spring scale; a hydraulic scale; and a pneumatic scale.

24. An apparatus according to claim 22, wherein the weighing arrangement of the captured solid and liquid bodily waste matter is arranged to support the receptacle within the toilet bowl in a spaced arrangement from the walls of the toilet bowl so as to measure the mass of the captured solid and liquid bodily waste matter.

25-30. (canceled)

31. A system for capturing bodily waste matter in a toilet bowl of a toilet, comprising:
a user interface for receiving user input for identifying a user of the toilet; and
an apparatus for capturing bodily waste matter deposited into a toilet bowl.

32. A system according to claim 31, wherein the apparatus for capturing bodily waste matter comprises :
a receptacle adapted to fit within a toilet bowl so as to releasably capture solid and liquid bodily waste matter within the toilet bowl, the receptacle including a sealable aperture arranged to provide an outlet for release of the solid and liquid bodily waste matter captured within the receptacle; and
a weighing arrangement for measuring the mass of the captured solid and liquid bodily waste matter.

33. A system according to claim 31, further comprising means for measuring a parameter of captured bodily waste matter.

34. A system according to claim 33, further comprising a database for storing the measured parameter on a record associated with the identified user.

35-36. (canceled)

37. A system according to claim 31, further comprising one or more adaptors configured to enable the apparatus to be fitted within one or more different toilet bowls having different dimensions.

38. (canceled)

39. A method of recording a measured parameter of bodily waste matter captured within a toilet bowl for a particular user, comprising:
identifying a user of an apparatus for capturing bodily waste matter in a toilet bowl;
receiving a measured parameter associated with bodily waste matter captured in the apparatus; and
storing said measured parameter in a memory, preferably in a database on a record associated with the identified user.

40. A method according to claim 39, wherein the apparatus comprises:
a receptacle adapted to fit within a toilet bowl so as to releasably capture solid and liquid bodily waste matter within the toilet bowl, the receptacle including a sealable aperture arranged to provide an outlet for release of the solid and liquid bodily waste matter captured within the receptacle; and
a weighing arrangement for measuring the mass of the captured solid and liquid bodily waste matter.

41-44. (canceled)

45. An apparatus according to claim 22, wherein the test apparatus comprises at least one of: a test strip, such as a multi-test strip that is manually retrievable; and at least one electronic probe, preferably wherein the measured parameter is manually or remotely retrievable from the at least one electronic probe.
